(19) United States
(12) Patent Application Publication
(10) Pub. No.: US 2018/0296624 A1
(43) Pub. Date: Oct. 18, 2018

(54) PREPARATIONS OF ROSA LAEVIGATA FRUIT EXTRACTS AND USES THEREOF FOR REGULATION OF CD36, APOA1, AND SRB1

(71) Applicant: TCI CO., LTD, Taipei (TW)

(72) Inventors: Yung-Hsiang Lin, Taipei (TW); Yu-Ting Lin, Taipei (TW)

(21) Appl. No.: 15/953,832

(22) Filed: Apr. 16, 2018

(30) Foreign Application Priority Data
Apr. 17, 2017 (TW) 106112805

(51) Int. Cl. A61K 36/738; A61K 9/16

(52) U.S. Cl. CPC A61K 36/738; A61K 9/16; A61K 2236/333; A61K 2236/331; A61K 2236/33

(57) ABSTRACT

Provided is a method of a method of preparing a Rosa laevigata fruit extract which regulates CD36, ApoA1, and SRB1, including extracting the fruit of Rosa laevigata using water, alcohols, or mixtures of water and alcohols as solvents. Also provided is a method of regulating CD36, ApoA1, and SRB1, including administering to a subject a composition containing an effective amount of the Rosa laevigata fruit extract. By suppressing the gene expression of CD36 and enhancing the gene expression of ApoA1 and SRB1, the Rosa laevigata fruit extract may be used to regulate lipid metabolism and reduce liver lipid accumulation and blood lipid levels.

### CROSS-REFERENCE TO RELATED APPLICATION

This application claims priority of Taiwan patent application No. 106112805, filed on Apr. 17, 2017, the content of which is incorporated herein in its entirety by reference.

### BACKGROUND OF THE INVENTION

### 1. Field of the Invention

The present invention relates to methods of preparing Rosa laevigata fruit extracts and using the same. Particularly, the present invention relates to a method of preparing Rosa laevigata fruit extracts which regulate CD36, ApoA1, and SRB1, and a method of regulating lipid metabolism, reducing liver lipid accumulation, and reducing blood lipid levels by using the Rosa laevigata fruit extracts.

### 2. The Prior Art

Diets of high fat and high sugar content and lack of exercise drive modern people towards obesity and increase the risks of developing metabolism-related diseases such as diabetes, hyperlipidemia, cardiovascular diseases, and fatty liver, which ultimately threatens physical health severely. In view of the above, much of the medical research in recent years has focused on seeking ways to control sugar and lipid metabolisms in an attempt to reduce the occurrence of these metabolic diseases.

Hyperlipidemia is a condition in which the concentration of lipids in blood is higher than the normal value. Blood lipids include triglycerides and cholesterols. Cholesterols are further classified into high-density lipoprotein cholesterol (HDL-C) and low-density lipoprotein cholesterol (LDL-C) based on the type of lipoproteins they reside. High-density lipoprotein (HDL) removes excess cholesterol from the bloodstream and transports it to the liver for metabolism. Therefore, HDL cholesterol is commonly called “good cholesterol.” In contrast, LDL cholesterol is called “bad cholesterol” because low-density lipoprotein (LDL) easily penetrates the blood vessel wall and accumulates on the arterial wall, causing the blocked arteries and even inflammation of blood vessels, leading to atherosclerosis. Thus, the major strategy to prevent atherosclerosis is to reduce blood lipid levels, particularly the triglycerides and LDL cholesterol, and to modestly increase HDL levels.

Fatty liver disease is clinically diagnosed when the liver fat content exceeds 5% of the liver weight. Due to different causes, fatty liver disease is further divided into alcoholic fatty liver disease and nonalcoholic fatty liver disease (NAFLD). According to studies related to lipid metabolism, NAFLD has a high correlation with the occurrence of cardiovascular diseases or atherosclerosis. Given the high global prevalence of NAFLD (about 20%) and its close relationship with atherosclerosis, another key to prevent atherosclerosis is to inhibit fatty liver development.

Accordingly, in order to reduce the incidences of the aforementioned various metabolic diseases and atherosclerosis, which predicts high mortality, it is of great necessity to develop a composition that regulates lipid metabolism and suppresses lipid accumulation in the liver.

### SUMMARY OF THE INVENTION

As a result, the present invention provides a method of regulating cluster of differentiation 36 (CD36), which functions as a free fatty acid receptor, apolipoprotein A1 (ApoA1), and scavenger receptor class B member 1 (SRB1), including administering to a subject a composition containing an effective amount of a Rosa laevigata fruit extract, wherein the Rosa laevigata fruit extract is obtained by solvent extraction of a Rosa laevigata fruit. The solvent is water, alcohol, or a mixture of water and alcohol; the liquid-to-solid ratio of the solvent to the Rosa laevigata fruit is from 20:1 to 1:1; and the extraction is performed at a temperature from 50° C. to 100° C.

In one embodiment of the present invention, the Rosa laevigata fruit extract in the composition is at a concentration of at least 0.5 mg/ml, and the regulation of CD36, ApoA1, and SRB1 is accomplished by suppressing the gene expression of CD36 and enhancing the gene expression of ApoA1 and SRB1 in liver cells, leading to regulation of lipid metabolism.

In another aspect, the present invention provides a method of reducing liver lipid accumulation, including administering to a subject a composition containing an effective amount of the aforementioned Rosa laevigata fruit extract.

In one embodiment of the present invention, the Rosa laevigata fruit extract in the composition is at a concentration of at least 0.5 mg/ml, and the reduction in liver lipid accumulation is accomplished by suppressing the gene expression of CD36 and enhancing the gene expression of ApoA1 and SRB1 in liver cells.

In yet another aspect, the present invention provides a method of preparing a Rosa laevigata fruit extract which regulates CD36, ApoA1, and SRB1, including the step of extracting a Rosa laevigata fruit with a solvent to obtain the Rosa laevigata fruit extract, wherein the solvent is water, alcohol, or a mixture of water and alcohol, the liquid-to-solid ratio of the solvent to the Rosa laevigata fruit is from 20:1 to 1:1, and the extraction is performed at a temperature from 50° C. to 100° C.

In one embodiment of the present invention, the preparation method further includes centrifuging the Rosa laevigata fruit extract to obtain a supernatant, filtering the supernatant to obtain a filtrate of the Rosa laevigata fruit extract, concentrating the filtrate at a temperature from 45° C. to 70° C. under reduced pressure to obtain a concentrated product of the Rosa laevigata fruit extract, and drying the concentrated product by spray drying to obtain a powder of the Rosa laevigata fruit extract.

The Rosa laevigata fruit extract obtained by the preparation method of the present invention suppresses the gene expression of CD36 and enhances the gene expression of ApoA1 and SRB1 in liver cells, and thus may be exploited to regulate lipid metabolism and reduce liver lipid accumulation and blood lipid levels. Moreover, the Rosa laevigata fruit extract has the potential to prevent or inhibit the development of fatty liver. Therefore, the Rosa laevigata fruit extract of the present invention can be utilized to prepare a composition for regulating CD36, ApoA1, and SRB1 and reducing lipid accumulation in the liver.

The present invention is further described in the following examples, in reference to the accompanying drawings. It should be understood that the examples given below do not limit the scope of the invention, and that modifications can be made without departing from the scope of the appended claims.

### BRIEF DESCRIPTION OF THE DRAWINGS

The present invention will be apparent to those skilled in the art from the following detailed description of the preferred embodiments, with reference to the attached drawings, in which:

FIG. 1A is a micrograph showing the HepG2 cells not treated with the Rosa laevigata fruit extract and stained with oil red O (at 400× magnification);

FIG. 1B is a micrograph showing the HepG2 cells treated with the Rosa laevigata fruit extract and stained with oil red O (at 400× magnification);

FIG. 1C shows the relative oil red O content of the HepG2 cells treated with or without the Rosa laevigata fruit extract and stained with oil red O;

FIG. 2 shows the relative gene expression levels of CD36 in the HepG2 cells treated with or without the Rosa laevigata fruit extract;

FIG. 3 shows the relative gene expression levels of ApoA1 in the HepG2 cells treated with or without the Rosa laevigata fruit extract; and

FIG. 4 shows the relative gene expression levels of SRB1 in the HepG2 cells treated with or without the Rosa laevigata fruit extract.

### DETAILED DESCRIPTION OF THE PREFERRED EMBODIMENT

### Definition

The data provided in the present invention represent approximated, experimental values that may vary within a range of ±20%, preferably ±10%, and most preferably ±5%.

The present invention provides a method of preparing a Rosa laevigata fruit extract which regulates CD36, ApoA1, and SRB1, including the step of extracting a Rosa laevigata fruit with a solvent to obtain the Rosa laevigata fruit extract, wherein the solvent is water, alcohol, or a mixture of water and alcohol, the liquid-to-solid ratio of the solvent to the Rosa laevigata fruit is from 20:1 to 1:1, and the extraction is performed at a temperature from 50° C. to 100° C. The present invention also discloses the Rosa laevigata fruit extract may reduce liver lipid accumulation and blood lipid levels through regulating the expression of genes related to lipid metabolism, including suppressing the gene expression of CD36 and enhancing the gene expression of ApoA1 and SRB1 in liver cells. Moreover, the Rosa laevigata fruit extract has the potential to prevent or inhibit the development of fatty liver. Therefore, the Rosa laevigata fruit extract of the present invention can be utilized to develop methods of regulating CD36, ApoA1, and SRB1 and reducing liver lipid accumulation, including administering to a subject, such as an obese person, a composition containing an effective amount of a Rosa laevigata fruit extract.

### Methods and Materials

### Materials

Dulbecco's modified Eagle's medium (DMEM; catalog number: 12100-038), fetal bovine serum (FBS; catalog number: 10438-026), penicillin/streptomycin (catalog number: 15140-122), and phosphate buffered saline (PBS; catalog number: 14200-075) were purchased from Gibco. Oil red O (catalog number: #0-0625) and oleic acid (catalog number: #O1008) were purchased from Sigma-Aldrich. Formaldehyde (catalog number: TG1794-4-0000-72NI) and isopropanol (catalog number: PH-3101) were purchased from Echo chemical CO. Ltd. Bovine serum albumin (BSA; catalog number: AD0023) was purchased from Bio Basic Inc.

### Cell Culture

HepG2 cells (ATCC HB-8065) from human hepatocellular carcinoma were used for experiments described in the following examples. The HepG2 cells were cultured in the DMEM supplemented with 10% FBS and 1% penicillin/streptomycin at 37° C. under 5% CO₂.

### Preparation of BSA-Conjugated Oleic Acid

Firstly, 0.34 mM BSA in PBS and 0.2 mM of oleic acid in PBS were prepared. The oleic acid was first dissolved in 100% ethanol and then mixed with PBS. Next, the two solutions were mixed at 1:1 volume ratio for at least one hour at 37° C. The solution of BSA-conjugated oleic acid was obtained when the mixture was clarified.

### Oil Red O Staining

The change in the neutral lipid content of cells was measured by oil red O staining. Prior to staining, cells were washed twice with PBS and fixed with 10% formaldehyde for 30 minutes. The fixed cells were washed twice with PBS and rinsed with 50% isopropanol for 15 seconds. Thereafter, the cells were stained with oil red O dye dissolved in 60% isopropanol for 1 hour, and the excess dye was washed off with deionized water. After staining, the cells were directly examined by microscope (ZEISS Axio Vert. A1), or treated with 100% isopropanol for quantitative analysis of the dissolved dyes in cells. Statistical analysis based on Student's t-test was performed by using the Excel software.

### Gene Expression Analysis

The expression levels of genes involved in lipid metabolism were determined based on quantitative polymerase chain reaction (referred to as qPCR). In brief, ribonucleic acid (RNA) was isolated from cells by using an RNA extraction kit (Geneaid) according to the manufacturer's instructions. The isolated RNA was reverse transcribed to complementary deoxyribonucleic acid (cDNA) at 37° C. by using reverse transcriptase (SuperScript® III, Invitrogen). Thereafter, the cDNA was subjected to PCR amplification by using a qPCR kit (KAPA CYBR FAST qPCR Kit (2X), KAPA Biosystems) and specific primer sets (forward and reverse primers for the CD36, APOA1, and SCARB1 genes; SEQ ID NOs:1-6). The qPCR was performed with StepOnePlus™ Real-Time PCR Systems.

### Example 1

Preparation of Rosa laevigata Fruit Extract

First, the fruit of Rosa laevigata, which has a diameter of 2-6 cm, is washed, air-dried, and ground with a homogenizer. Next, the Rosa laevigata fruit homogenate is extracted using water, alcohol, or a mixture of water and alcohol as the solvent. The liquid-to-solid ratio of the solvent to the Rosa laevigata fruit homogenate is from 20:1 to 1:1. The extraction temperature ranges from 50° C. to 100° C., preferably from 70° C. to 90° C. In one embodiment, the time for extraction is from 0.5 to 3 hours.

After the abovementioned extraction, the Rosa laevigata fruit extract is cooled to room temperature and may be further processed by centrifugation at 800 to 1300 rpm for 5 to 10 minutes at 15° C. to 25° C. to obtain a supernatant. The supernatant may be filtered through a 400 mesh filter to remove residual solids. The filtered Rosa laevigata fruit extract may further be concentrated under reduced pressure (less than 1 atm) at 45° C. to 70° C. to obtain a concentrated product. In order to produce a solid form of Rosa laevigata fruit extract, the concentrated product of Rosa laevigata fruit extract may be subjected to spray drying to remove the solvent, thereby obtaining the powder of Rosa laevigata fruit extract.

### Example 2

Rosa laevigata Fruit Extract Reduces Lipid Accumulation in Liver Cells

In order to examine the effect of the Rosa laevigata fruit extract of the present invention on liver lipid content, the change in lipid content of human HepG2 cells was analyzed by oil red O staining after treatment with the Rosa laevigata fruit extract. HepG2 cells were first seeded in 6-well plates at 5×10⁵ cells/well and cultured at 37° C. Next, the cells were treated with DMEM containing 0.5 mg/ml Rosa laevigata fruit extract and 2% FBS. After 24 hours, the medium was discarded and replaced with DMEM containing BSA-conjugated oleic acid, 2% FBS, and 1% penicillin/streptomycin, and the cells were treated with 0.5 mg/ml Rosa laevigata fruit extract for 24 hours. Finally, the medium was removed, and the cells were washed with PBS and stained with oil red O. The HepG2 cells treated similarly but without the Rosa laevigata fruit extract were used as control.

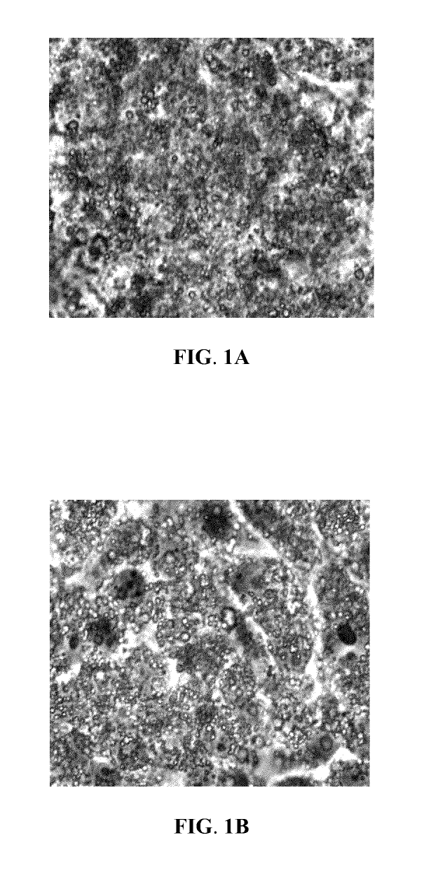

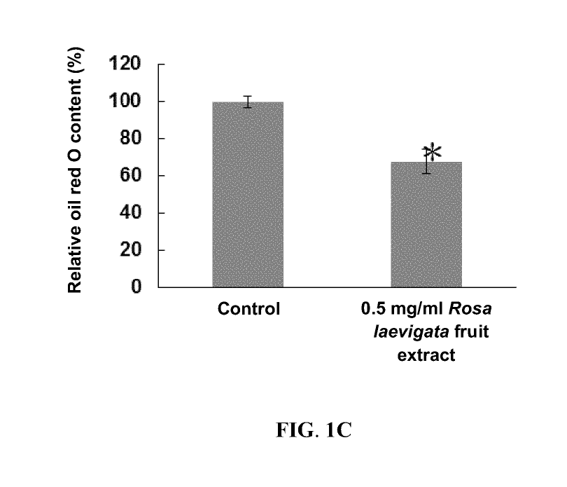

FIG. 1A and FIG. 1B, respectively, show the micrographs (at 400× magnification) of the control cells and the HepG2 cells treated with the Rosa laevigata fruit extract and stained with oil red O. By comparing FIG. 1A with FIG. 1B, it was found that a large amount of oil droplets stained in red were present in the control cells. In contrast, there was a significant reduction in the staining intensity of the HepG2 cells treated with the Rosa laevigata fruit extract. The oil red O-stained cells were further subjected to quantitative analysis of dye accumulation in cells. The results are shown in FIG. 1C, wherein the relative oil red O content (Y axis) is defined as the ratio (in percentage) of the measured oil red O content relative to that of the control cells, and * indicates p<0.01. According to FIG. 1C, there was significantly less oil red O accumulation in the HepG2 cells treated with the Rosa laevigata fruit extract. The results indicate that the Rosa laevigata fruit extract of the present invention is capable of reducing lipid accumulation in liver cells and has the potential to inhibit the development of fatty liver.

### Example 3

Rosa laevigata Fruit Extract Regulates the Expression of Genes Involved in Lipid Metabolism
3.1 Rosa laevigata Fruit Extract Suppresses the Gene Expression of CD36 in Liver Cells

In order to investigate the regulatory effect of the Rosa laevigata fruit extract of the present invention on lipid metabolic genes, the change in expression levels of lipid metabolic genes in HepG2 cells after treatment with the Rosa laevigata fruit extract was analyzed by qPCR. HepG2 cells were first seeded in 6-well plates at 1.5×10⁵ cells/well and cultured at 37° C. Next, the cells were treated with 0.5 mg/ml or 1 mg/ml Rosa laevigata fruit extract for 6, 24, or 48 hours, and then collected for qPCR analysis. The HepG2 cells treated with only the extraction solvent for preparing the Rosa laevigata fruit extract were used as control.

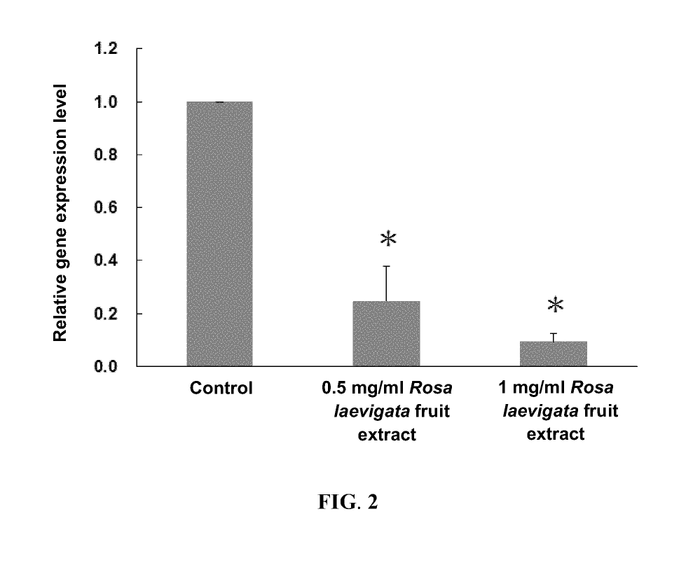

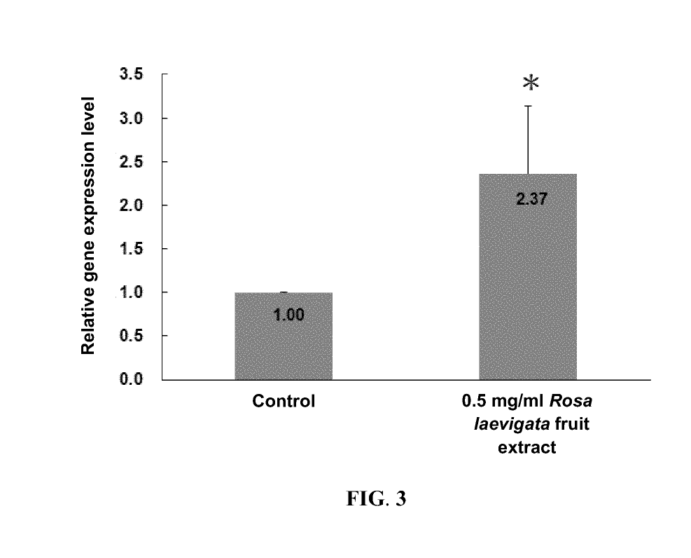

The relative gene expression levels of CD36 in HepG2 cells are shown in FIG. 2, wherein the relative gene expression level is presented as fold change relative to the measured gene expression level of CD36 in the control cells, and * indicates p<0.01. CD36 is a fatty acid translocase whose expression levels have been found by previous studies to be positively correlated with liver lipid accumulation and blood lipid levels. According to FIG. 2, the HepG2 cells treated with Rosa laevigata fruit extract exhibited much lower CD36 gene expression, approximately 25% of the CD36 expression level in control cells, and this reduction was more significant when the concentration of Rosa laevigata fruit extract was increased from 0.5 mg/ml to 1 mg/ml. The results indicate that the Rosa laevigata fruit extract of the present invention is able to inhibit the gene expression of CD36 in liver cells and thus reduce lipid accumulation in the liver and bloodstream.

3.2 Rosa laevigata Fruit Extract Enhances the Gene Expression of ApoA1 and SRB1 in Liver Cells

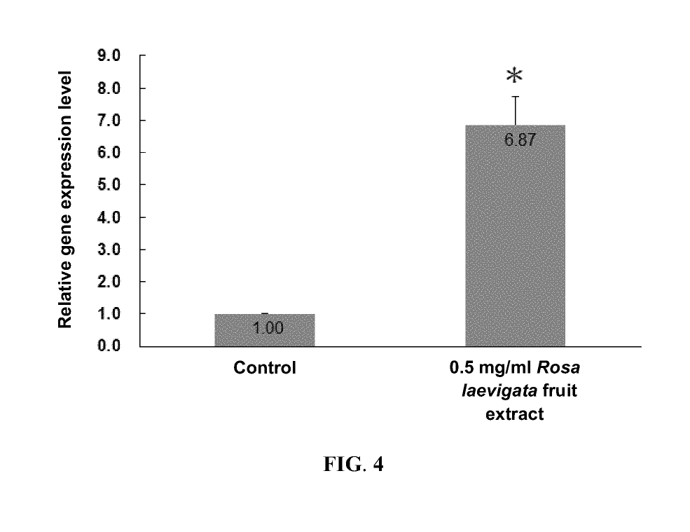

According to the experiments described in example 3.1, the relative gene expression levels of ApoA1 and SRB1 in HepG2 cells were determined and shown in FIG. 3 and FIG. 4, respectively. The relative gene expression levels are presented as fold change relative to the measured gene expression levels of ApoA1 and SRB1, respectively, in the control cells, and * indicates p<0.01. Previous studies have disclosed that ApoA1 is the primary protein constituent of HDL, and an increase in ApoA1 expression improves the synthesis of HDL in the liver, which in turn promotes the clearance of lipids such as cholesterol from the blood. SRB1 is an HDL receptor abundant in the liver that mediates the uptake of cholesterol from peripheral tissues and thus promotes the metabolism of cholesterol in the liver. According to FIG. 3 and FIG. 4, treatment of HepG2 cells with 0.5 mg/ml Rosa laevigata fruit extract significantly increased the gene expression of ApoA1 and SRB1 by 2.37-fold and 6.87-fold, respectively. The results indicates that the Rosa laevigata fruit extract of the present invention may promote HDL synthesis and cholesterol metabolism in the liver via increasing the hepatic gene expression of ApoA1 and SRB1, thereby modulating lipid metabolism and reducing blood lipid levels and liver lipid accumulation.

In conclusion, the Rosa laevigata fruit extract of the present invention that is obtained by solvent extraction with water, alcohol, or a mixture of water and alcohol suppresses the gene expression of CD36 and enhances the gene expression of ApoA1 and SRB1 in liver cells, resulting in regulation of lipid metabolism and reduction in liver lipid accumulation and blood lipid levels. Moreover, the Rosa laevigata fruit extract has the potential to prevent or inhibit the development of fatty liver. Therefore, the Rosa laevigata fruit extract of the present invention can be utilized to prepare a composition for regulating CD36, ApoA1, and SRB1 and reducing lipid accumulation in the liver. The composition may include a

What is claimed is:

1. A method of regulating cluster of differentiation (CD36), apolipoprotein A1 (ApoA1), and scavenger receptor class B member 1 (SRB1), comprising administering to a subject a composition containing an effective amount of a Rosa laevigata fruit extract, wherein the Rosa laevigata fruit extract is obtained by solvent extraction of a Rosa laevigata fruit, wherein the solvent is water, alcohol, or a mixture of water and alcohol, the liquid-to-solid ratio of the solvent to the Rosa laevigata fruit is from 20:1 to 1:1, and the extraction is performed at a temperature from 50° C. to 100° C.

2. The method of claim 1, wherein the Rosa laevigata fruit extract suppresses gene expression of the CD36.

3. The method of claim 1, wherein the Rosa laevigata fruit extract enhances gene expression of the ApoA1.

4. The method of claim 1, wherein the Rosa laevigata fruit extract enhances gene expression of the SRB1.

5. The method of claim 1, wherein the Rosa laevigata fruit extract in the composition is at a concentration of at least 0.5 mg/ml.

6. The method of claim 1, wherein the Rosa laevigata fruit extract regulates lipid metabolism in the subject.

7. A method of reducing liver lipid accumulation, comprising administering to a subject a composition containing an effective amount of a Rosa laevigata fruit extract, wherein the Rosa laevigata fruit extract is obtained by solvent extraction of a Rosa laevigata fruit, wherein the solvent is water, alcohol, or a mixture of water and alcohol, the liquid-to-solid ratio of the solvent to the Rosa laevigata fruit is from 20:1 to 1:1, and the extraction is performed at a temperature from 50° C. to 100° C.

8. The method of claim 7, wherein the Rosa laevigata fruit extract suppresses gene expression of the CD36.

9. The method of claim 7, wherein the Rosa laevigata fruit extract enhances gene expression of the ApoA1.

10. The method of claim 7, wherein the Rosa laevigata fruit extract enhances gene expression of the SRB1.

11. The method of claim 7, wherein the Rosa laevigata fruit extract regulates lipid metabolism in the subject.

12. A method of preparing a Rosa laevigata fruit extract which regulates CD36, ApoA1, and SRB1, comprising the step of extracting a Rosa laevigata fruit with a solvent to obtain the Rosa laevigata fruit extract,
wherein the solvent is water, alcohol, or a mixture of water and alcohol, the liquid-to-solid ratio of the solvent to the Rosa laevigata fruit is from 20:1 to 1:1, and the extraction is performed at a temperature from 50° C. to 100° C.

13. The method of claim 12, further comprising centrifuging the Rosa laevigata fruit extract to obtain a supernatant, and filtering the supernatant to obtain a filtrate of the Rosa laevigata fruit extract.

14. The method of claim 13, further comprising concentrating the filtrate at a temperature from 45° C. to 70° C. under reduced pressure to obtain a concentrated product of the Rosa laevigata fruit extract.

15. The method of claim 14, further comprising drying the concentrated product by spray drying to obtain a powder of the Rosa laevigata fruit extract.


SEQUENCE LISTING (6 sequences)

SEQ ID NO: 1 - DNA, 25 bp, Artificial sequence
  Forward primer for CD36 gene
atatggtgtgctagacatcagcaaa

SEQ ID NO: 2 - DNA, 19 bp, Artificial sequence
  Reverse primer for CD36 gene
ggttgacctgcagccgttt

SEQ ID NO: 3 - DNA, 23 bp, Artificial sequence
  Forward primer for APOA1 gene
ctgtgtacgtggatgtgctcaaa

SEQ ID NO: 4 - DNA, 21 bp, Artificial sequence
  Reverse primer for APOA1 gene
caggccctctgtctccttttc

SEQ ID NO: 5 - DNA, 21 bp, Artificial sequence
  Forward primer for SCARB1 gene
actccgactctgggctcttca

SEQ ID NO: 6 - DNA, 18 bp, Artificial sequence
  Reverse primer for SCARB1 gene
ggcctccgggctgtagaa